(19) United States
(12) Patent Application Publication
(10) Pub. No.: US 2026/0042738 A1
(43) Pub. Date: Feb. 12, 2026

(54) SYNTHESIS OF CAPROLACTAM FROM MUCONIC ACID AND OTHER BIO-DERIVED SUBSTRATES

(71) Applicant: Alliance for Sustainable Energy, LLC, Golden, CO (US)

(72) Inventors: Hyunjin MOON, Lakewood, CO (US); Ali CHAMAS, Lakewood, CO (US); Gregg Tyler BECKHAM, Golden, CO (US)

(21) Appl. No.: 19/295,187

(22) Filed: Aug. 8, 2025

Related U.S. Application Data
(60) Provisional application No. 63/681,022, filed on Aug. 8, 2024.

(51) Int. Cl. C07D 223/10; B01J 23/20; B01J 23/46; C08G 69/14

(52) U.S. Cl. CPC C07D 223/10; B01J 23/20; B01J 23/462; C08G 69/14

(57) ABSTRACT

Disclosed herein are methods and compositions of matter and processes for synthesizing caprolactam for nylon-6 production form muconic and adipic acids. A transition metal catalyst (typically ruthenium) on an acidic support (typically niobia) was synthesized to catalyze the amidation and cyclization to caprolactam.

### CROSS-REFERENCE TO RELATED APPLICATIONS

This application claims priority under 35 U.S.C. § 119 to U.S. provisional patent application No. 63/681,022 filed on Aug. 8, 2024, the contents of which are hereby incorporated in their entirety.

### CONTRACTUAL ORIGIN

The United States Government has rights in this invention under Contract No. DE-AC36-08GO28308 between the United States Department of Energy and Alliance for Sustainable Energy, LLC, the Manager and Operator of the National Renewable Energy Laboratory. This invention was made under CRADA CRD-23-23605 between Patagonia, Inc. and Alliance for Sustainable Energy, LLC. The United States Government has certain rights in this invention.

### BACKGROUND

Bio-based caprolactam has been proposed and demonstrated through multiple pathways to date, most of which suffer from yields that are too low to be commercially viable. These pathways include routes through 1) the (biological or chemo-catalytic) deamination and ring closure of the amino acid, L-lysine, 2) through biological production of 6-aminocaproic acid and (biological or chemo-catalytic) ring closure thereof, and 3) through the conversion of the C6 diolefinic diacid, cis,cis-muconic acid (hereafter muconate), among other routes.

Among these known pathways, the production of lysine is a commercial, at-scale process today, and the yields of this amino acid from glucose are between 40-50 mol %. Moreover, the chemo-catalytic conversion of lysine to caprolactam is already known. However, the lysine-to-caprolactam process has not been developed into a commercially viable strategy. In the current state-of-the-art approach, this route suffers from the loss of an —NH₂ group in the conversion of lysine to caprolactam, the use of low temperatures (−5° C.) in the process, and excessive base consumption (8 equiv. of KOH), which generates substantial K₂SO₄ waste.

### SUMMARY

In an aspect, disclosed herein methods and processes for the synthesis of caprolactam from adipic or muconic acid.

Other objects, advantages, and novel features of the present invention will become apparent from the following detailed description of the invention when considered in conjunction with the accompanying drawings.

### BRIEF DESCRIPTION OF THE DRAWINGS

FIG. 1 depicts muconate production from glucose and xylose in corn stover-derived hydrolysate sugars in a fed-batch bioreactor.

FIG. 2 depicts cellulosic sugars that can be converted into cis,cis-muconic acid through engineered microbes, such as Pseudomonas putida KT2440. As previously demonstrated in batch reaction systems, this intermediate can be converted into caprolactam catalytically, either directly (top pathway) or through hydrogenation to adipic acid (bottom pathway), but over-hydrogenation to hexamethyleneimine in both cases is a driver of low yields (<555%) previously achieved by others.

FIG. 3 depicts a prophetic embodiment wherein a flow reactor is initiated with a two-fixed bed configuration comprising columns containing TiO₂—P25 for an amidation reaction at from about 170-200° C. and then a Ru/d-o-Nb₂O₅ support for a hydrodeoxygenation reaction at from about 200-225° C. In an embodiment, the amidation reaction would be conducted first to inhibit decarboxylation/decarbonylation of adipic acid by Ru. In an embodiment, the substrates provided to the two-fixed bed flow reactor are adipic acid, t-butanol and NH₃—H₂O. In an embodiment, hydrogen gas is introduced into the two-fixed bed flow reactor after the amidation reaction and before the hydrodeoxygenation reaction. In an embodiment, caprolactam is produced using the two-fixed bed reactor. Ru/d-o-Nb₂O₅ refers to a composite material where ruthenium (Ru) is supported on a defective niobium pentoxide (Nb₂O₅) support, likely with oxygen defects (d-o).

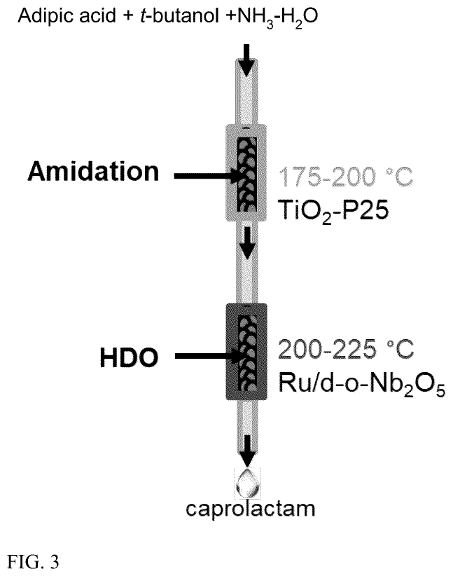

FIG. 4 depicts an embodiment of caprolactam yield percentage over time using Ru/d-o-Nb₂O₅ or Ru/Nb₂O₅ supports from a provided solution of adipic acid, t-butanol and NH₃—H₂O using conditions of H₂ at 20 bar at 225° C. after a first amidation reaction and then a second hydrodeoxygenation reaction.

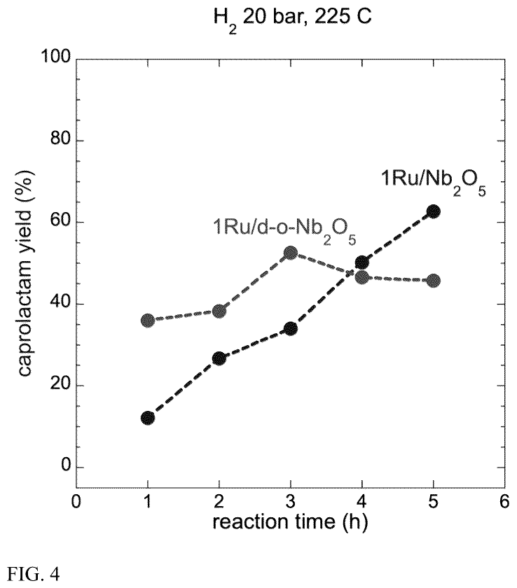

### DETAILED DESCRIPTION

Disclosed herein are systems, methods and compositions of matter for the conversion of bio-based muconic acid to bio-based caprolactam at theoretical yield for use in nylon-6 synthesis. In an embodiment, methods disclosed herein result in or are useful for formulating an optimal heterogeneous catalyst for muconic acid conversion to caprolactam; designing and optimizing a corresponding, flow-based catalytic process; and developing a downstream purification approach to achieve monomer-grade caprolactam purity. Processes disclosed herein include, in an embodiment, a process for synthesizing caprolactam for nylon-6 production from muconic and adipic acids. In an embodiment, a transition metal catalyst (typically ruthenium) on an acidic support (typically niobia) is synthesized to catalyze the amidation and cyclization to caprolactam and a novel sequential flow reactor is designed and optimized.

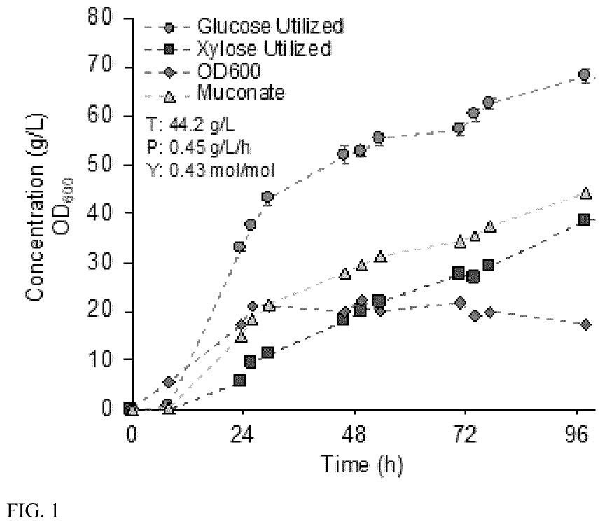

The caprolactam synthesis pathway through muconate is more promising for several reasons described below. Muconate can be produced from cellulosic sugars as well as from lignin-derived aromatic compounds via lignin valorization and from plastics-derived aromatic compounds such as benzoic acid from polystyrene waste or terephthalic acid from polyethylene terephthalate waste. In the cellulosic sugars-to-muconate pathway, the current state-of-the-art production is 44.2 g/L, 0.45 g/L/hr, and a molar yield of 43% from glucose and xylose on corn stover-derived hydrolysate (see FIG. 1). We have done this work at the 150 L scale, and in an embodiment the reactions may be scaled up to the 1,000 L or greater level. In an embodiment, a sugar, arabinose, may be used by non-naturally occurring strains for carbon and energy, which should increase productivity substantially. The techno-economic analysis and life cycle assessment shows substantial promise for bio-based muconate to be cost competitive with fossil-based adipic acid at a corresponding greenhouse gas emissions reduction of about 80%.

Muconate to caprolactam has been demonstrated previously. Others have used batch reactors and (mostly) off-the-shelf heterogeneous catalysts to demonstrate that hydrogenation of muconate followed by introduction of NH₃ which leads to reasonable yields of caprolactam of up to about 55 mol %. However, prior work has not included appreciable catalyst development pursued in a mechanistic context, and no chemistry has been previously done in a flow-based reactor system. From a process perspective, especially relative to lysine, muconate can be produced at similar molar yields than lysine from sugars and it can be made at 100% molar yield from aromatic substrates (e.g., from lignin or aromatic plastics). Additionally, muconate to caprolactam is a more atom-efficient pathway from a nitrogen perspective than lysine, wherein in the latter case, 2 molar equivalents of NH₃ are required and 1 molar equivalent is lost, whereas for muconate, only 1 molar equivalent of NH₃ is required.

In an embodiment, methods are disclosed herein for developing a heterogeneous catalyst and a corresponding flow-based process for the conversion of muconate to caprolactam. In another embodiment, methods for catalyst formulation and process development are disclosed.

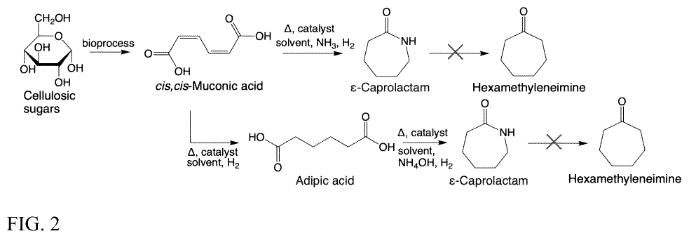

The conversion of muconate to caprolactam is a selectivity challenge. Namely, muconate conversion to caprolactam is clearly tractable, but a catalytic process can readily over-hydrogenate to hexamethyleneimine (HMI), as depicted in FIG. 2. Here, methods are disclosed to synthesize a library of monometallic and bimetallic catalysts on weak acid supports including titania and zirconia. In an embodiment, catalysts generated using methods disclosed herein are characterized using TPR, TPD, N2-BET, SEM, TEM, XRD, etc. and screened with (non-protic) solvent combinations in a high-throughput batch reaction system. In an embodiment, benign solvents and catalytic metals with low environmental impacts are used.

Based on batch reaction screening results, at least three catalysts may be transitioned into a flow-based process in an existing flow reactor. Using methods disclosed herein, conversion of muconic acid, and yield and selectivity of the target product and off-target products are increased when compared to prior methods. In an embodiment, disclosed herein is a selectivity descriptor for caprolactam production achieved through the systematic study of accessible catalyst design parameters (and measurements), solvent properties, and reaction engineering variables. In a prophetic embodiment, yields of caprolactam achieved are greater than 90%.

In an embodiment, polymerization reactions and the properties of polymers created by using methods and compositions disclosed herein are determined to (e.g., MW, thermal properties) ensure that the bio-based nylon-6 generated is equivalent to incumbent nylon-6 used today.

In an embodiment, Ru catalysts supported on Nb₂O₅, and TiO₂ were active in hydrodeoxygenation reactions with adipamide while MoOₓ, NiMo, and beta-MoC showed lower yields comparatively.

### Materials and Methods

Adipamide hydrodeoxygenation: In a stainless-steel Parr reactor (75 mL), 0.69 mmol of adipamide, 75 mg of Ru catalyst (e.g. Ru/Nb₂O₅, Ru/TiO₂), and 14 mL of t-butanol were added. To that, 1 mL of NH₄OH or NH₃ (3-6 bar) was added to the reactor. The reactor was pressurized with H₂ gas (4-20 bar), and the temperature was increased to the desired reaction temperature (180-225° C.). After the reaction, Parr reactors were quenched in ice, and the catalyst was removed by filtration using 0.2 μm filter. The product solution was diluted with water to analyze the yields of caprolactam and other by-products.

Adipic acid amidation to caprolactam: The reaction was conducted under the same conditions, with 0.69 mmol of adipic acid used in place of adipamide.

Adipic acid amidation: In the stainless-steel Parr reactor (75 mL), 0.68 mmol of adipic acid, 75 mg of catalyst (e.g., Nb₂O₅, TiO₂, etc), and 14 mL of t-butanol were added. For the nitrogen source, 7-8 mmol of nitrogen sources (NH₄OH, NH₃-isopropyl alcohol, or NH₃-methanol solution) was added to the reactor. The reactor was pressurized with 20 bar of N₂, and the temperature was increased to the desired reaction temperature (125-225° C.). After the reactions, the Parr reactor was quenched in ice. t-butanol was removed by using a rotavap at 50° C. under 40 mbar, and formic acid aqueous solution was added to dissolve all the products. The final solution was diluted with water to analyze the yields of adipamide and 6-amino-6-oxohexanoic acid.

The foregoing disclosure has been set forth merely to illustrate the invention and is not intended to be limiting.

We claim:

1. A method for synthesizing caprolactam from muconic acid comprising using a transition metal catalyst on an acidic support to catalyze the amidation and cyclization of muconic acid to caprolactam.

2. The method of claim 1 wherein the caprolactam is used for synthesizing nylon-6.

3. The method of claim 1 wherein the transition metal catalyst comprises ruthenium.

4. The method of claim 1 wherein the acidic support comprises niobia.

5. The method of claim 1 wherein the caprolactam is produced at greater than 55 mol percent from the muconic acid.

6. A method for synthesizing caprolactam from adipic acid comprising using a transition metal catalyst on an acidic support to catalyze the amidation and cyclization of the adipic acid to caprolactam.

7. The method of claim 6 wherein the caprolactam is used for synthesizing nylon-6.

8. The method of claim 6 wherein the transition metal catalyst comprises ruthenium.

9. The method of claim 6 wherein the acidic support comprises niobia.

10. The method of claim 6 wherein the caprolactam is produced at greater than 55 mol percent from the adipic acid.

11. A process for synthesizing caprolactam from muconic acid comprising using a transition metal catalyst on an acidic support to catalyze the amidation and cyclization of muconic acid to caprolactam.

12. The process of claim 11 wherein the caprolactam is used for synthesizing nylon-6.

13. The process of claim 11 wherein the transition metal catalyst comprises ruthenium.

14. The process of claim 11 wherein the acidic support comprises niobia.

15. The process of claim 11 wherein the caprolactam is produced at greater than 55 mol percent from the muconic acid.

16. A process for synthesizing caprolactam from adipic acid comprising using a transition metal catalyst on an acidic support to catalyze the amidation and cyclization of the adipic acid to caprolactam.

17. The process of claim 16 wherein the caprolactam is used for synthesizing nylon-6.

18. The process of claim 16 wherein the transition metal catalyst comprises ruthenium.

19. The process of claim 16 wherein the acidic support comprises niobia.

20. The process of claim 16 wherein the caprolactam is produced at greater than 55 mol percent from the adipic acid.
